(19) United States
(12) Patent Application Publication
(10) Pub. No.: US 2009/0093702 A1
(43) Pub. Date: Apr. 9, 2009

(54) DETERMINING AND IDENTIFYING CHANGES IN THE POSITION OF PARTS OF A BODY STRUCTURE

(76) Inventors: Fritz Vollmer, Munchen (DE); Martin Leidel, Miesbach (DE); Ingmar Thiemann, Munchen (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 12/243,463

(22) Filed: Oct. 1, 2008

Related U.S. Application Data
(60) Provisional application No. 60/977,528, filed on Oct. 4, 2007.

(30) Foreign Application Priority Data
Oct. 2, 2007 (EP) 07117756

(51) Int. Cl. A61B 5/05

(52) U.S. Cl. 600/407

(57) ABSTRACT

The present invention relates to a method for determining and/or identifying a change in the position of a part of a body structure relative to a marker device by means of a data processing device, comprising the steps of:
providing pre-operative data of the body structure which comprises information concerning the pre-operative position of the part of the body structure relative to the marker device;
providing intra-operative data of the body structure which comprises information concerning the intra-operative position of the part of the body structure relative to the marker device; and
in order to determine whether a change in position has occurred, automatically determining—on the basis of the pre-operative position of the part and the intra-operative position of the part—whether the position of the pre-operative part differs from the position of the intra-operative part and/or automatically displaying the pre-operative position of the part and the intra-operative position of the part, such that a different position can be identified.

### RELATED APPLICATION DATA

This application claims priority of U.S. Provisional Application No. 60/977,528 filed on Oct. 4, 2007, which is incorporated herein by reference in its entirety.

Operations are preferably planned by the physician by means of so-called pre-operative data (for example, nuclear spin data or CT data). Particularly when operating on soft parts (for example, the brain or liver), it can occur that the soft parts (which represent an example of a body structure) shift relative to other body structures (for example, bones) during the operation. The present invention relates in particular to navigation-assisted surgery in which pre-operative data concerning the body structure is provided which describes the relative position of the body structure relative to markers. However, as mentioned, the position of at least a part of the body structure can shift relative to the markers during the operation. This shift or displacement can be identified using inter-operative analysis methods (for example, ultrasound or x-ray recordings) which detect both the at least one part of the body structure and the markers.

It is an object of the invention to automatically determine changes in position or to make it easier for the operator to identify a change in position.

The above object is solved by the subjects of the independent claims. Advantageous developments follow from the dependent claims.

The method in accordance with the invention preferably serves to determine and/or identify a change in the position of a part of a body structure relative to a marker device. A body structure can in particular include soft parts and bones. The head represents one example of a body structure. A marker device is preferably fixedly connected to the body structure (for example, rigid parts of the body structure such as bones). The marker device comprises for example a reference star and/or a number of marker elements (marker spheres). The marker elements can for example be designed to be active, such that they emit rays and/or waves (for example light, in particular infrared light). They can also be designed to be passive, such that they reflect waves and/or rays, i.e. in particular reflect light (for example, infrared light) which is for example emitted from a ray source and/or wave source (for example, a light source). The passively reflected waves and/or rays or the actively emitted waves and/or rays are preferably detected by a detection device which can thus detect the position of the marker device. The marker elements in particular assume a particular position relative to each other.

In accordance with the invention, pre-operative data and intra-operative data of the body structure is provided. The pre-operative data differs from the intra-operative data in particular in that it was acquired earlier. The pre-operative data is in particular more comprehensive in its information content than the intra-operative data. A pre-operatively applied medical analysis method, such as for example a nuclear spin or three-dimensional x-ray CT method, is usually provided with a longer period of time in order to collect information. The spatial resolution of the data which describes the spatial structure of the body structure is in particular finer in the case of pre-operative data than in the case of intra-operative data. The pre-operative data can also differ from the intra-operative data in that it was acquired from various medical analysis devices. Ultrasound diagnosis apparatuses can for example be used intra-operatively, and the data thus acquired can be processed together with pre-operatively acquired nuclear spin data using the method in accordance with the invention.

Preferably, both the pre-operative data and the intra-operative data describes the position of at least one particular part of the body structure (or also of the entire body structure) relative to the marker device. On the basis of the pre-operative data and the intra-operative data, it is preferably determined whether the position of the at least one particular part of the body structure described by the pre-operative data is different from the position of the (same) part of the body structure described by the intra-operative data. This determination is in particular made automatically and is then in particular preferably combined with automatically outputting notifying information if a change in the position of the part between the pre-operative situation and the intra-operative situation, i.e. a different position, is determined. A corresponding determination can of course also be made for a number of different parts of the body structure. In the following, the invention is described—purely by way of example—on the basis of a part (or sub-structure) which changes its position in the time between the pre-operative data being captured and the intra-operative data being captured.

Alternatively or additionally, at least the part of the body structure such as follows from the intra-operative data and the pre-operative data is preferably displayed, in particular automatically, in accordance with the invention. It is preferably displayed such that an operator can easily identify whether the position of the pre-operative part differs from the position of the (corresponding) intra-operative part. In particular, the position of the pre-operative part and the position of the (corresponding) intra-operative part are for example shown superposed or next to each other or one below the other, wherein in the latter cases, the display is preferably underlaid with a scale so as to be able to more easily determine a shift in position visually. In particular when the two parts are superposed in the display, they can be displayed in different colors. Automatically determining whether a change in position has occurred, i.e. whether the positions differ, can in particular be combined with automatically displaying the parts. The combination can in particular be such that those parts which have experienced a change in position are visually highlighted (for example, in color or by blinking).

The pre-operative part and/or the intra-operative part are preferably represented by so-called (anatomical) objects which result from the pre-operative data and/or intra-operative data by applying image processing algorithms (for example, object extraction, smoothing or edge detection).

The image data corresponding to the pre-operative data and the intra-operative data is preferably scaled and preferably output in the same coordinate system, wherein in particular the markers which are not necessarily shown assume the same position in the two coordinate systems, such that the operator can compare the position when the image data is displayed. As already stated above, this comparison is preferably facilitated by displaying a scale.

If a determination is automatically made as to whether the position of the pre-operative part is different from the position of the intra-operative part, this can be based on an operator designating both the pre-operative part and the intra-operative part in the image data, for example interactively, for example by circumscribing them with a mouse. The pre-operative part and/or the intra-operative part are preferably determined automatically.

It is assumed that the operator has planned a particular region within the pre-operative data as being relevant to the operation. This particular region for example contains a pre-operative part, i.e. for example a tumor in the brain. The operator can then determine the pre-operative part in this way, by defining its boundaries, for example using a mouse. Alternatively, image processing algorithms can for example extract a coherent structure, for example from a roughly designated region, wherein said structure then represents the pre-operative part of the body structure (or a pre-operative sub-structure), the shape and/or change in position of which are to be determined or identified in accordance with the invention. The corresponding intra-operative part is preferably determined automatically on the basis of the intra-operative data and on the basis of the determined pre-operative part.

The correspondence between the pre-operative part and the intra-operative part can for example be based on the shape of the pre-operative and/or intra-operative part. The pre-operative data and intra-operative data therefore preferably includes information concerning the shape of the part of the body structure. For example, a curved or round shape of the pre-operative part may be derivable from the pre-operative data, and a corresponding curved or round shape can then be sought in the intra-operative data, so as to automatically determine the intra-operative part corresponding to the pre-operative part. This can of course also be performed for a number of parts. Matching and/or pattern recognition methods can in particular be used for this purpose, in order to determine corresponding parts on the basis of the pre-operative data and the intra-operative data. Additionally or alternatively, the results of a medical analysis which is used in order to acquire the intra-operative data can for example be simulated on the basis of the pre-operative data. This simulated result can then for example be compared with the intra-operative data, again using matching and/or pattern recognition methods, so as to identify corresponding parts of the body structure.

Alternatively and in particular additionally, it is possible to refer to the positional information when automatically determining corresponding parts. Probabilities can in particular be calculated, in accordance with which the correspondence is more probable, the more similar the position of the potentially corresponding parts relative to the position of the marker device. These principles of probability, for example the principles of the maximum entropy method. can in particular be used when determining a correspondence based on shape and/or position. wherein it is possible to refer not only to information based on a position relative to the marker device but also based on a position relative to other parts (for example, bone portions or landmarks).

Elastic deformation methods based on maximizing reciprocal information can in particular be used, wherein entropy is one of the possibilities for information over-determination which is maximized in the case of elastic registrations.

Notifying information preferably provides information concerning not only the change in position, in particular the extent of a change in position, but alternatively and in particular additionally also concerning a change in shape which is determined by comparing the pre-operative shape with the intra-operative shape. The notifying information can in particular be configured such that it also provides information concerning the extent of the change in shape, for example by visually highlighting (for example in color or by blinking) portions of the part which have been subjected to a significant change in shape.

Image data is preferably displayed on the basis of the pre-operative data. In accordance with one embodiment of the invention, this display can be configured or changed such that the position and/or shape of the pre-operative part is changed in accordance with the position and/or shape of the intra-operative part, wherein in particular the remaining image data remains unaltered. such that for example a shift in the position or change in the shape of the pre-operative part relative to the remaining (pre-operatively determined) body structure results and can be identified on a display. This approach is particularly advantageous when the pre-operative data is more true to detail and in particular when it is easy to identify whether or not the pre-operative part is then situated closer to locations which are critical to the operation. For example, it is thus possible to identify whether or not a tumor has changed its position such that it has then moved to within the vicinity of a sensitive part of the brain (for example, the speech centre). The pre-operatively acquired information is preferably supplemented with the intra-operative data, in particular when the intra-operative analysis method differs from the pre-operative ones and thus detects other aspects and (physical/chemical) parameters of the body structure.

It is of course also possible in accordance with the invention for a shift in position to be displayed and viewed for a number of parts.

In particular with the intra-operative analysis methods, it can be the case that these methods only detect a component part of a sub-structure of the body structure, for example only the edge of a sub-structure (for example, a tumor). In an ultrasound examination, for example, it can occur that only a part of the edge of a tumor is detected and can thus be identified as an intra-operative part on the basis of the intra-operative data. The intra-operative part (for example, the edge portion of the tumor) is thus only a component part of a sub-structure (for example, a tumor). In accordance with the invention, information is preferably provided not only concerning a change in the position of the component part of a sub-structure, but—based on the assumption that the entire sub-structure has changed its position along with the change in the position of the component part—also concerning the change in the position of the sub-structure. Information is for example provided in this respect such-that in an image representation based on the pre-operative data, not only the change in the position of the component part of a sub-structure is displayed but also the change in the position of the entire sub-structure. Alternatively or additionally, the sub-structure which—given the aforesaid assumption—has altered its position can be visually highlighted (for example in a particular color or by blinking). In particular, a corresponding change in the position of the sub-structure is calculated in accordance with the invention on the basis of the change in the position of the intra-operative part relative to the pre-operative part, and this change in position is displayed using notifying information. The notifying information can for example be a display of the pre-operative sub-structure in its pre-operative position and another display of the intra-operative sub-structure in its intra-operative position, wherein a scale is additionally displayed in each case, so that the operator can identify the extent of the change in position. Alternatively, a superposed visual representation of the pre-operative position of the sub-structure and the intra-operative position of the sub-structure can be selected. Alternatively, it is for example also possible to display the pre-operative position of the sub-structure only or the intra-operative position of the sub-structure only, and to illustrate how significant the calculated change in position turns out to be, using a color code.

By displaying the pre-operative part and the intra-operative part and/or the pre-operative sub-structure and the intra-operative sub-structure as cited above, it is in particular also possible to identify changes in shape which have resulted between the pre-operative situation and the intra-operative situation (data acquisition).

In addition to the intra-operative part and the pre-operative part or the intra-operative sub-structure and the pre-operative sub-structure, it is also possible to display (anatomical) objects derived from them, by applying image processing algorithms.

The change in the shape of the sub-structure is preferably calculated on the basis of particular properties of the sub-structure, at least for those portions of the sub-structure for which no intra-operative data is available. When calculating the change in shape, it may in particular be assumed that the sub-structure has particular physical properties which are for example expressed in a certain plasticity or elasticity of the sub-structure. Alternatively or additionally, it may be assumed that particular geometric properties of the sub-structure have not changed or have only changed to a certain extent between the time the pre-operative data is captured and the time the intra-operative data is captured. It may for example be assumed that the volume, or an area which includes or surrounds the sub-structure, has remained constant or has only changed to a particular extent of for example less than 20% or less than 10%. Based on this assumption, it is then also possible to calculate the probable intra-operative shape of the sub-structure for those portions for which no information is available on the basis of the intra-operative data. A particular elasticity constant or a particular range can for example be assumed for a sub-structure (for example, a tumor), and it is also possible to assume that forces or the effects of pressure on the tumor can change to a particular extent intra-operatively, and thus for example to assume an upper limit for deformation. The portion of the sub-structure for which intra-operative data has been acquired and for which information concerning the shape of said portion is thus available can of course also act as an boundary condition. If, for example, on the basis of this information and assuming a constant volume, a deformation of the sub-structure occurs which cannot be reconciled with the assumed ranges for the elasticity constant and the effects of force and/or pressure, a warning notification can be output. This can then indicate that the calculated shape of the sub-structure outside of the portion secured by the intra-operative data may not be correct.

The invention also relates to a program which performs the aforesaid method on a computer. The program includes in particular software interfaces for recording the pre-operative data or intra-operative data, in order to then be able to process them in accordance with the method.

The present invention is also directed to a device which is designed to determine and/or identify a change in the position of the part of the body structure relative to the marker device. The device can be part of a medical navigation system, such as is used in particular in image-guided operations (image-guided surgery). The device preferably comprises a marker detection device for detecting the marker device, in particular for intra-operatively detecting the marker device. Furthermore, the device can also comprise the medical analysis device (or, for example, receive data from it) which is intra-operatively used for acquiring the intra-operative data. This medical analysis device is preferably designed such that it can detect both the position of the part of the body structure and the position of the marker device. A data storage device, such as for example a hard disc or ROM, is preferably also provided in order to store the pre-operative data of the body structure, and for example receives the pre-operative data via data interfaces. Data interfaces are for example interfaces with the Internet or removable storage drives. The data processing device can for example be a conventional computer.

One advantage of the invention is that an operator (for example, a surgeon) can identify possible shifts in parts of a body structure as compared to the previously performed planning based on the pre-operative data, by looking at the displayed image or comparing displayed images. wherein one is for example based on the pre-operative data and the other is based on the intra-operative data. An overlapping representation of the pre-operative data and the intra-operative data is preferred. In the case of ultrasound images, the previously planned boundaries of the parts of the body structure (the object boundaries) are advantageously displayed in the intra-operative ultrasound image, so as to display shifts.

In addition to ultrasound diagnostics, it is for example also possible to use so-called iMRI (diffusion-weighted MRI images), in order for example to visualize nerve tracts.

An iMRI is an integrated MR apparatus which can be used during an operation. It can be used for normal MR recordings or also for diffusion-weighted imaging or diffusion tensor imaging (DTI). From a diffusion-weighted series of MR recordings, information can be acquired which enables the diffusion properties of the tissue and therefore in particular the path of nerve fibers in the brain to be displayed.

The intra-operatively detected nerve tracts represent examples of a part of a body structure. In accordance with the invention, they can be compared, in particular automatically, with pre-operative (usually higher-resolution) data concerning the path of nerve tracts. In accordance with the invention, quantitatively assessing the shift or deformation is in particular facilitated.

Thus, in accordance with the invention, data is acquired intra-operatively, for example by means of navigated three-dimensional ultrasound measuring apparatuses or intra-operatively acquired CT/MRI data, in order to in particular detect the position and/or shape of a part of the body structure (for example, a tumor, lesion, nerve fibers, etc.). The part of the body structure can in particular be pre-operatively determined within the framework of a plan.

The boundaries of the part of the body structure are advantageously determined in particular by using modality-inherent features. For instance, (tumor) tissue is represented differently in different modalities (when using different measuring techniques). Some tumors are highly visible in ultrasound; some are not at all visible. Tumor boundaries may be imaged in ultrasound better than the tumor itself, etc. Brain tissue is differently visible in MR depending on the recording modality (T1/T2). Nerve tracts can for example also be calculated for DTI (diffusion tensor imaging) from pre-operative and intra-operative MRI image data. The advantage is that of determining the deformation during the operation, on the basis of the nerve tracts. The nerve fibers before and during the operation can advantageously be compared. and any shifts in tissue can therefore be deduced. DTI can equally be produced using a sufficiently equipped intra-operative MR as pre-operatively. Since the fiber connections to and/or from functional areas are important, information concerning their path is advantageous during the operation. One advantage of the invention is that of deducing general shifts in tissue from the visualization of changes in the nerve fibers.

It is in particular possible to identify parts of the body structure, more specifically to identify which parts of the body structure represented by the intra-operative data correspond to parts represented by pre-operative data, as follows:
1. The shape of the pre-operative part (i.e. the part resulting from the pre-operative data) can be used as a pattern or template, in order to identify and find a corresponding part in the intra-operative data.
2. If the intra-operative imaging conditions are known, in particular the intra-operatively used medical analysis methods, then the pattern or template can be correspondingly configured beforehand on the basis of their specific properties. For example in ultrasound recordings, shadowing of the side of a part facing away from the ultrasound receiver occurs under certain conditions. Shadowing may but need not occur. Such shadowing can be simulated, so as to facilitate an optimized pattern (or template) for identifying and locating a corresponding part in the intra-operative data. In other words, on the basis of the properties of the intra-operatively used medical imaging methods (analysis methods), it is possible to generate a pattern for which a better match with the part derived from the intra-operative data results than for the pre-operative part. It is thus possible to increase the probability of identifying a correspondence between the intra-operative part and the pre-operative part.
3. Edge detection filters can for example be applied to the pre-operatively and/or intra-operatively acquired data, so as to identify the boundaries of the part of the body structure (the object boundaries). By assuming elastic object properties, it is possible to deduce the entire object from (parts of) the edge.
4. For automatically determining the change in position in particular, the pattern (pattern of the part or pattern of the object) can in particular be three-dimensionally moved, in particular in the space defined by the intra-operative data, until a match between the pattern of the part and the intra-operative part is optimized, in particular until a match between the edges or boundaries of the pattern of the part and the intra-operatively detected edges or boundaries is maximized. If this is the case, then the shift in the position of the part of the body structure is determined on the basis of this. In a second step, a possible change in shape can then be determined, for example by continuously changing the shape of the pattern of the part until an optimum match of the shapes results. By means of a parametric, elastic model, it is in particular possible to change the shape iteratively, until a maximum overlap of the shapes is achieved.

The method in accordance with the invention, in particular the program or device in accordance with the invention, can (then) display the change in the position of the part of the body structure (the object of the data processing), in particular with respect to the pre-operative position of the part of the body structure. In particular, deformations in the part of the body structure (for example, a pre-operatively determined image object as compared to the intra-operatively determined image object) can also be visualized. From the visualization of deformed nerve tracts, for example, the operator can more easily deduce the intra-operative deformation in essential brain structures.

In accordance with the invention, the part of the body structure (for example, the pre-operatively planned image object) can in particular be automatically shifted to the current position in the registered three-dimensional patient coordinate system. The patient is registered, since a marker device attached to the patient is preferably detected in accordance with the invention using a navigation system, wherein said marker device has preferably also been detected by the intra-operative and also pre-operative analysis methods. It is thus in particular possible to display the current position of an instrument relative to the intra-operative position of the part of the body structure, wherein it is possible for the display to refer to the spatially finer information of the pre-operative data when displaying the part of the body structure and simultaneously for the position of the part of the body structure displayed in the patient coordinate system to still correspond to the currently, intra-operatively determined position.

In addition to the aforementioned methods, other physical parameters—in particular of the tissue—can also be used, for example for determining the elasticity, in order to determine a correspondence between the intra-operative part and the pre-operative part and in particular to detect the current shape of the intra-operative part. Vibrography (J Ultrasound Med 2005; 24: 985-992) represents an example of this. Vibrography allows elastic properties of the brain and in particular of a tumor tissue to be detected and thus enables information to be captured which does not follow from ultrasound images. Thus, in accordance with the invention, vibrography can be combined with an ultrasound diagnosis, so as to be able to better determine the boundaries of the part of the body structure (for example, the boundaries of the tumor) intra-operatively. In particular, vibrography allows indistinctly delineated parts of body structures (i.e. for example, indistinctly delineated tumor regions) to be displayed when it is not possible to clearly differentiate between the tumor and the brain. Thus, if the data from various medical analysis methods is combined in accordance with the invention, in order to obtain in particular intra-operative data from this combination, it is then possible in accordance with the invention to more precisely determine parts of the body structure and in particular of sub-structures.

Intra-operative data acquisition is advantageously configured such that the essential structures—which are crucial to the operation and have in particular been defined pre-operatively—are in particular or exclusively detected by the intra-operatively used medical analysis device. The method in accordance with the invention enables the operator, in particular a surgeon. to identify the movement of the important structures (parts of the body structure) as compared to the situation during pre-operative planning. The invention advantageously quantifies the change in position which occurs from the planning to the intra-operative situation, and the operator can use this quantified information in order to modify the current approach as compared to the planned approach. The data thus acquired can also be used for a planned, post-operative radiation treatment. The present invention advantageously visualizes deformations or change in the size of parts of the body structure for the operator. In particular, the shape of a previously planned object can be corrected. Color coding can in particular be used to visualize changes in shape. Advantageously, planned objects (pre-operative parts) are automatically displayed and fitted to the current position when visualizing the part of the body structure. Parts which have a rigid relative relationship to the pre-operative data, i.e. which in particular have a rigid spatial relationship to a marker device attached to the body structure and are intra-operatively identified by the medical analysis device, are advantageously used to refine registration, in particular the registration of instruments.

Bends and deformations in nerve tracts, which are for example measured by intra-operatively used DTI, are advantageously used to estimate deformations in the brain.

The intra-operatively used medical analysis methods are advantageously used to supplement pre-operatively acquired data. Thus, a combination of pre-operatively acquired data and intra-operatively acquired data is advantageously displayed, wherein the position of a part or a sub-structure of the pre-operative data and/or the position of an object which is derived from the pre-operative data and represents the part or sub-structure is/are changed in accordance with the invention, in order to match the intra-operative situation. The combination of ultrasound data. Doppler sonography data and vibrography data can for example provide additional information concerning tumor boundaries which did not follow from pre-operatively acquired MRI images. It is thus possible to ensure that the operation captures the entire tumor. Thus, by using different measuring techniques (ultrasound, MR, CT, etc.) and recording parameters (ultrasound B mode, vibrography or MR T1, T2, DTI), it is possible to acquire more information.

The forgoing and other features of the invention are hereinafter discussed with reference to the drawings.

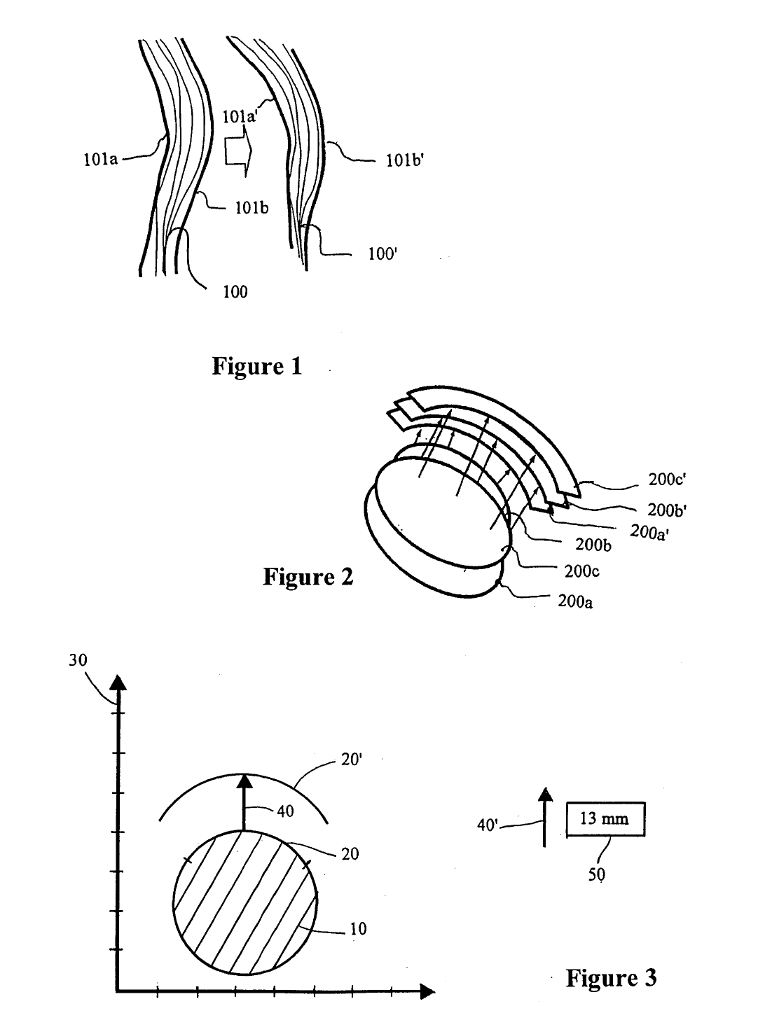

FIG. 1 is a schematic diagram showing an exemplary change in the shape of nerve tracts.

FIG. 2 illustrates the boundaries of a part of a sub-structure.

FIG. 3 is an exemplary visualization of a change in position.

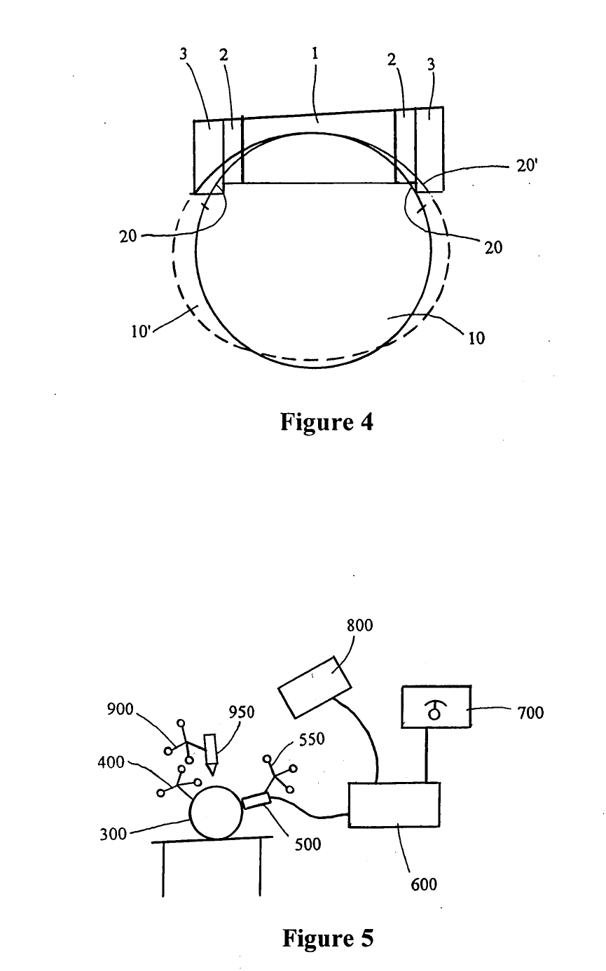

FIG. 4 illustrates an exemplary overlapping representation of a pre-operative object and an intra-operative object.

FIG. 5 schematically shows an exemplary device in accordance with the invention.

In the following, a detailed description of embodiments discloses other advantages and features of the invention. Different features of different embodiments can be combined with each other.

When using different analysis techniques to generate images of a body structure, boundaries of parts or sub-structures of the body structure can be measured, or calculated from the image data obtained for example by means of DTI, depending on the imaging technique. These properties of the sub-structure are preferably used pre-operatively in order to automatically (for example. by means of edge detection) or manually define a three-dimensional object, which can correspond to the pre-operative part or the pre-operative sub-structure, in the pre-operative data set. The boundaries of a tumor can for example be pre-operatively defined on the basis of an MRI data set, or the boundaries of nerve tracts can for example be pre-operatively defined on the basis of a DTI data set.

FIG. 1 schematically shows the change in the shape of nerve tracts 100 and 100′, wherein 100 represents the pre-operative situation and 100′ represents the intra-operative situation. The thick black lines 101 and 101′ highlight the pre-operative and intra-operative boundaries of the nerve tracts, which can for example be acquired from the pre-operative data using edge detection and result intra-operatively for example from DTI images. The boundaries are preferably also acquired pre-operatively using DTI. Intra-operative edge detection along bundles of fibers which have already been calculated and segmented helps in particular in performing the fit in an algorithmically simpler way. Fitting the edges can be calculated more simply and more quickly. Preferably, the change as a whole is assumed to be an elastic deformation, in an analogous way to the deformed edges. Edge detection is in particular a means for fitting the object in a simplified way. In ultrasound in particular, it enables basic object properties to be obtained, for fitting. Edge detection is optionally applied to the intra-operative data. Pre-operative segmenting is preferably, though not compulsorily, performed using (more complex) other (standard) methods (atlas-based, region growing, thresholding, magic wand, etc.) or manually.

As may be seen, the path and in particular the curvature of the pre-operative boundaries 101a and 101a′, and 101b and 101b′, are similar. The curvature properties can thus be used in order to automatically identify the boundaries in the intra-operative data set. It is also possible, when automatically identifying the boundaries, to refer to the position of the boundaries 101a relative to 101b, and to the absolute position of the nerve tracts relative to a marker device.

Overall, the similarity between the shapes of the intra-operative part as compared to the pre-operative part can be used to automatically identify objects. The boundaries of a part of the body structure can in particular be highlighted for an operator, for example by means of edge detection, in order make it easier for him to identify the corresponding object in the image which is based on intra-operative data.

If the intra-operative part which corresponds to the pre-operative part has been determined, i.e. if an anatomical object has been extracted from the intra-operative data set, a possible shift in position can then be identified.

FIG. 2 shows a case in which the pre-operatively used medical analysis technique differs from the intra-operatively used analysis technique. 200a, 200b and 200c show a pre-operatively determined sub-structure of a tumor, which for illustrative reasons is displayed in a simplified form as discs of the tumor which collectively form a three-dimensional tumor object. Dividing an object into a number of component objects (layers), for example disc-shaped component objects, also makes it easier to identify a change in shape (see below). It is, however, purely optional, and it is also possible to use a single three-dimensional object.

The selected representation of layers can also take into account the image modality, when the data is recorded in layers using ultrasound, MR and CT.

The boundaries of a part of this sub-structure (which, in this case, is shadowed in the ultrasound) are identified in the ultrasound and schematically shown in FIG. 2 by boundary surfaces 200a′, 200b′ and 200c′. The boundary surfaces 200a′, 200b′ and 200c′ represent examples of an intra-operative part of the body structure, which is in turn part of a sub-structure. In this case, the sub-structure represents the boundary surface of the tumor 200. Thus, on the basis of the pre-operative data, one of the aforementioned standard methods, or for example an edge detection algorithm, is for example advantageously applied for the purpose of segmenting, so as to determine the boundary surface of the discs 200a, 200b and 200c. This boundary surface is then the sub-structure. At least a part of this boundary surface is then to be matched to the boundary surfaces 200a′ and 200b′ and 200c′. If a shift in the position of the tumor between the pre-operative situation and the intra-operative situation has occurred, then the aforesaid match can be achieved by shifting the discs 200a, 200b and 200c, as indicated in FIG. 2 by arrows, until the boundaries of the discs fall within the boundary surfaces 200a′, 200b′ and 200c′. An associated displacement and/or deformation of the tumor 200 can thus be quantitatively detected. The magnitude of the displacement follows from the length of the arrows shown in FIG. 2. A deformation can for example be determined when a different displacement results for the individual discs. A deformation can also be derived from a deviating curvature of the boundary surfaces, as explained in more detail further below.

Before the boundary surfaces ascertained from the pre-operative data by segmenting (or other standard methods, for example edge extraction) are fitted to the intra-operative boundary surfaces, a result of the intra-operatively used medical analysis method is advantageously simulated. In an ultrasound examination of a tumor, for example, it is known that shadowing of the part of the tumor which faces away from and not towards the ultrasound probe can occur. Thus, as an intermediate step with respect to the method shown in FIG. 2, corresponding ultrasound signals can be simulated from the shape and position data of the tumor 200 resulting from the nuclear spin data. The simulated ultrasound signals are then similar to the crescent-shaped boundary surfaces 200a′ and 200b′ and 200c′ shown in FIG. 2. These crescent-shaped, simulated boundary surfaces need only then be matched to the boundary surfaces 200a′, 200b′ and 200c′ by changes in shape and position.

The aforesaid method has been described to the effect that an object is divided into two-dimensional discs. The method cited can of course also be performed without dividing into discs, i.e. using three-dimensional pre-operative objects which represent parts or sub-structures of the body structure, in order to determine which change in position or shape leads to the best match between the pre-operative object and the three-dimensional intra-operative object. A linear transformation, which in particular includes a translation and/or rotation, is preferably used for changing the position.

The change in shape and/or position is preferably visualized. One example of visualizing a change in position is shown in FIG. 3. An optional scale 30 is indicated, in order to enable the operator to estimate the change in position. A pre-operative object 10, which corresponds to a pre-operative sub-structure, is displayed. An intra-operative object, which corresponds to an intra-operative part of the body structure, has the reference sign 20′. The position of the object 10 and the position of the object 20′ differ. A match between a portion 20 of the boundary of the object 10 and the object 20′ can be achieved by means of a transformation which is displayed on the screen using an arrow 40. The portion 20 is the pre-operative part of the body structure which corresponds to the intra-operative part 20′. As shown on the right in FIG. 3, the length of the shift vector 40 can also be displayed on the screen as a separate arrow 40′. The value 50 or amount of the shift—in the given example, 13 mm—can also be displayed separately in a region of the screen.

FIG. 4 shows an example of an overlapping representation of a pre-operative object 10 and an intra-operative object 20′. The object 10 can already have been shifted by a transformation prior to the representation in FIG. 4, in order to achieve at least a partial superposition or overlap between the object 10 and the object 20′ (corresponding to an at least partial superposition between the pre-operative part 20 and the intra-operative part 20′) and so make a change in shape easier for an operator to identify. Preferably, a maximum possible overlap of the edge or volume is aimed for. The change in position in FIG. 4 can then additionally be displayed using an arrow 40′ or a value of the change in position, as in FIG. 3.

When visualizing a deformation such as is shown in FIG. 4, color charts are preferably used which show different colors depending on the extent of deformation. The region 1 can for example be green, so as to indicate that no deformation has been detected in this region 1. The region 2 can be yellow, in order to indicate that a slight deformation has been detected in this region. Lastly, the region 3 can be red, in order to indicate that a significant deformation has been detected in this region. Additionally, an object 10′ can be automatically calculated. The edge of the object 10′ is indicated in FIG. 4 by a broken line and also includes the portion 20′. The object 10′ can for example be calculated by assuming that the object 10 can be elastically deformed and subject to the condition that the portion 20 is completely matched to the portion 20′.

It is thus possible to highlight deformations in the part of a body structure, and thus differences in shape between pre-operative and intra-operative objects, on the screen using color coding. Such changes in shape can for example occur due to a shift in the brain or can be explained by the fact that the medical analysis techniques differ, such that for example regions of the tumor are intra-operatively detected which were not identified pre-operatively. By highlighting the differences, for example using color codes (or blinking at different frequencies), it is thus possible to indicate to the surgeon that there are deviations from his plan. This makes it easier for example for the surgeon to capture the entire tumor.

FIG. 5 schematically shows the design of a device in accordance with the invention. A patient's head 300 is provided with a marker device 400 which is for example fixedly attached to the skull. An ultrasound device 500 is in contact with the patient's head, in order to acquire intra-operative data. The ultrasound device 500 also has a marker device 550 attached to it, in order to be able to navigate it. The ultrasound device 500 is connected to a data processing device 600 which produces notifying information, for example in accordance with FIG. 3 or FIG. 4, on a monitor 700.

A marker detection device 800 allows the marker device 400 and the marker device 900, which is attached to the instrument 950, to be detected, and passes the detection signals to the data processing device 600. The latter can thus in particular also calculate the position of the instrument or the position of the tip of the instrument relative to the intra-operative position of the part of the body structure (for example, the object 20 in FIG. 3 or FIG. 4). If, for example, it is assumed that the positions are displayed in accordance with FIG. 4, then the surgeon can identify whether his instrument lies in the critical regions 3 in which a deformation has occurred and in which he thus has to deviate from his plan. Also, on the basis of the object 10, he can identify the space which the sub-structure (the tumor) approximately occupies, even though only a part can be directly derived from the intra-operative data. By taking into account the change in the position of the sub-structure between the pre-operative, planned situation and the intra-operative situation, it is possible to reduce the risk of damaging healthy tissue and to increase the probability of completely removing the diseased tissue.

1. A method for determining and/or identifying a change in the position of a part (20; 100) of a body structure relative to a marker device (400) by means of a data processing device, comprising the steps of:
providing pre-operative data of the body structure which comprises information concerning the pre-operative position of the part (20; 100) of the body structure relative to the marker device (400);
providing intra-operative data of the body structure which comprises information concerning the intra-operative position of the part (20′; 100′) of the body structure relative to the marker device (400); and
in order to determine whether a change in position (40) has occurred, automatically determining - on the basis of the pre-operative position of the part (20; 100) and the intra-operative position of the part (20′; 100′)—whether the position of the pre-operative part (20; 100) differs from the position of the intra-operative part (20′; 100′) and/or automatically displaying the pre-operative position of the part (20; 100) and the intra-operative position of the part (20′; 100′), such that a different position can be identified.

2. The method according to claim 1, wherein if it is determined that the position differs, corresponding notifying information (FIG. 3) is automatically output.

3. The method according to claim 1, wherein if it is determined that the position differs, the change in position is calculated and notifying information (FIG. 3) is output which provides information concerning the extent of the change in position.

4. The method according to claim 1, wherein an image is displayed on the basis of the pre-operative data, and at least one of the following steps is performed:
the position of the pre-operative part and the position of the intra-operative part is displayed in the image (FIG. 3); and/or
a new position of the pre-operative part is calculated on the basis of the change in the position of the part, and the pre-operative part is displayed in the image in its changed position (FIG. 4), as an alternative to or in addition to the original position; and/or
the extent of the change in position is visually highlighted in the image (FIG. 3).

5. The method according to claim 1, wherein on the basis of the intra-operative data and the pre-operative data, the intra-operative part (20′; 100′) corresponding to the pre-operative part (20; 100) is automatically determined and/or a correspondence is automatically verified.

6. The method according to claim 5 the preceding claim, wherein:
the pre-operative data of the body structure comprises information concerning
the pre-operative shape of the part of the body structure;
the intra-operative data of the body structure comprises information concerning the intra-operative shape of the part of the body structure;
on the basis of a comparison between the pre-operative shape of the part and the intra-operative shape of the part, an automatic determination and/or verification is made as to whether the pre-operative part corresponds to the intra-operative part.

7. The method according to claim 5, wherein in order to automatically determine whether the pre-operative part (20; 100) corresponds to the intra-operative part (20′; 100′), at least one of the following steps is performed:
a matching and/or pattern recognition method and/or maximum entropy method is used, in which the pre-operative part is compared with the intra-operative part; and/or
on the basis of the pre-operative data of the body structure, a result of an intra-operative analysis method used for acquiring the intra-operative data is modeled, and the result is compared with the intra-operative data; and/or
transformations for iteratively maximizing an overlap between the pre-operative part and the intra-operative part are used.

8. The method according to claim 1, wherein a change in the shape of the part is determined on the basis of the pre-operative shape of the part (20) and the intra-operative shape of the part (20′), and wherein the change in shape is in particular highlighted by means of colors (FIG. 4).

9. The method according to claim 6, wherein on the basis of the similarity between the pre-operative position and the intra-operative position of the part and/or the similarity between the positional relationship between at least two pre-operative parts (101a, 101b) and the positional relationship between at least two intra-operative parts (101a′, 101b′), an automatic determination and/or verification is made as to whether the pre-operative part (100) corresponds to the intra-operative part (100′).

10. The method according to claim 1, wherein:
on the basis of the pre-operative data, an identification is made as to whether the pre-operative part (20) is a component part of a sub-structure (10) of the body structure which is larger than and includes the pre-operative part (20); and
information is automatically provided (40) concerning the fact that the pre-operative position of the sub-structure (10) is different from the intra-operative position, if the pre-operative position of the part (20) is different from the intra-operative position of the part (20′).

11. The method according to claim 10, wherein:
if the position of the pre-operative part is different from the position of the intra-operative part, a change in position is automatically calculated (50) from the pre-operative position of the part and the intra-operative position of the part;
an intra-operative position of the sub-structure (10) is automatically calculated on the basis of the calculated change in position and the pre-operative position of the sub-structure; and
the intra-operative position of the sub-structure (10) is automatically displayed (FIG. 4).

12. The method according to claim 10, wherein a change in the shape of the part is determined on the basis of pre-operative shape of the part (20) and the intra-operative shape of the part (20′), and a changed shape of the sub-structure (10′) is determined on the basis of the calculated change in shape and the pre-operative shape of the sub-structure (10), and displayed.

13. The method according to claim 12, wherein geometric properties of the sub-structure are determined on the basis of the pre-operative data, these geometric properties are assumed to be invariable or only variable to a predetermined extent, and the change in the shape of the sub-structure is determined on the basis of this assumption.

14. The method according to claim 12, wherein in order to calculate the change in the shape of the sub-structure, it is assumed that the sub-structure can be elastically deformed.

15. A program which, when it is running on a computer or is loaded onto a computer, causes the computer to perform the method according to claim 1.

16. A device for determining and/or identifying a change in the position of a part of a body structure relative to a marker device which is fixed with respect to the body structure, comprising:
a medical analysis device (500) for capturing intra-operative data of the body structure which comprises information concerning the intra-operative position of the part (20′) of the body structure relative to the marker device (400);
a data storage device which stores pre-operative data of the body structure which comprises information concerning the pre-operative position of the part (20) of the body structure relative to the marker device (400);
a data processing device (600) on which the program according to the preceding claim is loaded or is running.

17. A navigation system, comprising:
a device according to claim 16;
an instrument (900) to which another marker device (950) is attached;
a marker detection device (800) for detecting the fixed marker device (400) and the other marker device (950) and for inputting detection signals based on the detection into the data processing device (600);
wherein the data processing device is designed to display the position of the instrument (950) relative to the intra-operative position of the part (20′) of the body structure on the basis of the detection signals, wherein in order to display the part (20′) or a sub-structure (10′) which includes the part, reference is at least also made to the extent of information in the pre-operative data, which is larger than that of the intra-operative data, while in order to determine the position and/or shape of the part (20′) of sub-structure (10′), reference is at least also made to the intra-operative data, even if the intra-operative data only contains information concerning the part (20′) and not the entire sub-structure (10′).
